(19) United States
(12) Patent Application Publication
(10) Pub. No.: US 2015/0065423 A1
(43) Pub. Date: Mar. 5, 2015

(54) RAPID ACTING INJECTABLE FORMULATIONS

(71) Applicant: Perosphere, Inc., Danbury, CT (US)

(72) Inventors: Bryan E. Laulicht, Danbury, CT (US); Sasha H. Bakhru, Danbury, CT (US); Solomon S. Steiner, Mount Kisco, NY (US)

(21) Appl. No.: 14/472,471

(22) Filed: Aug. 29, 2014

Related U.S. Application Data
(60) Provisional application No. 61/872,185, filed on Aug. 30, 2013.

(51) Int. Cl. A61K 9/00; A61K 31/21; A61K 47/10; A61K 38/28

(52) U.S. Cl. CPC A61K 9/0019; A61K 38/28; A61K 31/21; A61K 47/10
USPC 514/6.3; 514/6.5

(57) ABSTRACT

A rapid acting injectable formulation is provided comprising a therapeutic peptide and a vasodilatory agent. The therapeutic peptide has a molecular weight of greater than about 500 Daltons, and the vasodilatory agent is present in an amount effective to increase the absorption of the therapeutic peptide. A method of increasing absorption of a therapeutic peptide by using such a formulation is also provided.

### CROSS-REFERENCE TO RELATED APPLICATION

This application claims the benefit of U.S. Provisional Patent Application No. 61/872,185, filed Aug. 30, 2013, the entire disclosure of which is incorporated by reference herein.

### BACKGROUND OF THE INVENTION

1. Field of the Invention

This invention relates to rapid acting injectable formulations. In particular, it relates to rapid acting injectable formulations comprising a therapeutic peptide and a vasodilatory agent.

2. Description of Related Art

In healthy individuals, many peptide hormones have a characteristic spike in output followed by a rapid decline to baseline values. These spikes often signal important biological functions. Insulin, for example, is secreted virtually immediately on meal ingestion. In patients with insulin insufficiency and/or resistance, insulin must be administered to replace normal pancreatic function. The kinetics of insulin circulation is crucial to and inextricable from achieving glycemic control in insulin-dependent patients. Insulin signals the liver to cease the production and release of glucose into systemic circulation. Additionally, insulin signals liver, fat and skeletal muscle cells to absorb circulating glucose. Any time lag between the ingestion of a meal and signaling the liver to discontinue glucose production can lead to hyperglycemia. Chronic hyperglycemia is associated with severe health consequences including cardiovascular, central and peripheral neurological, renal and retinal damage. Additionally, the lag between the introduction of glucose into the bloodstream and the spike in insulin concentration is associated with weight gain. The lag between glucose ingestion and the bioavailability of insulin is a widespread problem for both bolus and continuous pump based treatments.

Currently available rapid acting injectables, for example, rapid-acting insulin injectables fail to adequately mimic the pharmacokinetic profiles of insulin in a non-diabetic. As a result, the incidence of hyperglycemia caused by the delay in onset of action from currently available injectable insulin and insulin analogs is increased as compared to healthy individuals. Further, the incidence of severe hypoglycemia due to the mismatch in timing between insulin activity and the end of meals is also increased as compared to healthy individuals.

Additionally, parathyroid hormone (PTH) and its active fragments (e.g. PTH1-34) cause bone generation when spiked; however, they can signal bone resorption if the circulation lasts for too long at too low a level. Therefore, a rapid acting PTH or PTH fragment would improve the therapeutic benefit of PTH formulations.

Calcitonin can also be used to improve bone growth in patients experiencing untoward bone loss. Creating a rapid acting formulation of calcitonin may improve its bioactivity in a similar fashion to PTH.

Human growth hormone (HGH) injections would also benefit from a rapid acting formulation. Changes in HGH levels have the greatest impact on growth. Therefore a more rapid acting HGH formulation than is currently available could improve treatment efficacy.

There is a longstanding unmet clinical need for a broad-based strategy for increasing blood circulation and/or vasodilating at the site of sub-cutaneous injections to increase the rate of absorption of injectable therapies.

### SUMMARY OF THE INVENTION

The present invention is directed to rapid acting injectable formulations comprising a therapeutic peptide and a vasodilatory agent. The therapeutic peptide has a molecular weight of greater than about 500 Daltons, and the vasodilatory agent is present in an amount effective to increase the absorption of the therapeutic peptide.

In one embodiment, the rapid acting injectable formulation is administered sub-cutaneously.

In another embodiment, the vasodilatory agent is combined with a surfactant, a co-solvent or a combination thereof.

Another embodiment of the present invention is a method of increasing absorption of a therapeutic peptide by using a rapid acting injectable formulation comprising a therapeutic peptide and a vasodilatory agent. The therapeutic peptide has a molecular weight of greater than about 500 Daltons, and the vasodilatory agent is present in an amount effective to increase the absorption of the therapeutic peptide.

### BRIEF DESCRIPTION OF THE DRAWINGS

FIG. 1 is a plot depicting the HPLC chromatogram for an embodiment of the present invention.

FIG. 2 is a plot depicting the HPLC chromatogram for an embodiment of the present invention with 20% aqueous solution of PEG3350.

FIG. 3 is a plot depicting the average blood glucose concentration [mg/dL] as function of time for a comparative insulin lispro formulation (solid black line with squares) and for an embodiment of the present invention (dashed black line with diamonds) in a diabetic swine.

FIG. 4 is a plot depicting the insulin lispro IU/mL plasma concentration as function of time for a comparative insulin lispro formulation (solid black line with squares) and for an embodiment of the present invention (dashed black line with diamonds) in a diabetic swine.

### DETAILED DESCRIPTION OF THE INVENTION

An embodiment of the present invention is related to a rapid acting injectable formulation comprising a therapeutic peptide and a vasodilatory agent with an increased absorption of the therapeutic peptide. The therapeutic peptide has a molecular weight of greater than about 500 Daltons, and the vasodilatory agent is present in an amount effective to increase the absorption of the therapeutic peptide. Another embodiment of the present invention is a method of increasing absorption of a therapeutic peptide by using a rapid acting injectable formulation.

In one embodiment, the rapid acting injectable formulation is administered sub-cutaneously. A subcutaneous injection of therapeutic fluids, drugs, proteins, and other compounds is typically performed using syringe, pen injectors and other devices.

As used herein, “increase the absorption of the therapeutic peptide” means an increase in the absorption of the therapeutic peptide by at least approximately five percent in the area under the plasma concentration versus time curve.

Blood glucose levels can be measured by known techniques, such as by chemical methods including, but not limited to glucose in the presence of an oxidizing or reducing agent yielding a color change such as the Folin-Wu, Benedict's, Nelson-Somogyi, neocuproine, Shaeffer-Hartman-Somogyi, Hagedorn-Jensen, by a condensation method such as orthotoluidine, anthrone, enzymatic methods such as glucose oxidase, Saifer-Gerstenfeld, Trinder, Kodak Ektachem, glucometer, hexokinase methods, or continuous glucose monitoring methods.

Plasma concentration of insulin or its analog can be measured by known techniques, such as insulin enzyme-linked immunosorbent assay (ELISA), or radioimmunoassay (RIA).

In an embodiment of the present invention, any therapeutic peptide that has a molecular weight of greater than about 500 Daltons can be used. As used herein, the therapeutic peptide may be a single peptide or a complex of peptides. Thus, if the therapeutic peptide exists as a complex of two or more therapeutic peptides, the molecular weight of the therapeutic peptide will be the sum of the molecular weights of the therapeutic peptides that form the complex. Preferably, the therapeutic peptide has a molecular weight of about 500 Daltons to about 100 kiloDaltons, more preferably, about 750 Daltons to about 50 kiloDaltons, even more preferably, about 1 kiloDalton to about 40 kiloDaltons. Non-limiting examples of suitable therapeutic peptide include insulin, insulin analog, parathyroid hormone (PTH), glucagon, C-peptide, calcitonin, parathyroid hormone, human growth hormone, and incretins.

Insulin and its analogs are used in patients with Type I and II diabetics for the treatment of hyperglycemia. PTH and its active fragments (e.g. PTH1-34) are used in patients with osteoporosis and hypoparathyroidism. Glucagon is used in patients with Type I and II diabetics for the treatment of hypoglycemia, including rescue indications and maintenance by way of a bi-hormonal pump. C-peptide is used in patients with Type I and II diabetics for restoring pancreatic output. Calcitonin is used in patients with postmenopausal osteoporosis, hypercalcaemia, Paget's disease, bone metasteses, and phantom limb pain. Human growth hormone (hGH) is used in patients with growth disorders. Incretins are used in patients with Type I and II diabetics.

In a preferred embodiment, the therapeutic peptide is insulin or insulin analog. Human insulin consists of 51 amino acids and has an A and a B chain linked by disulfide bonds. The molecular weight of human insulin is 5.8 kDa. Insulin is synthesized in and secreted by beta cells in the islets of Langerhans within the pancreas. In the body, insulin is stored as a hexamer, the form in which it is most stable. The monomeric form of insulin is more reactive and diffuses more quickly in the body than the hexameric form and so the monomer form is advantageous with respect to activity.

Non-limiting examples of the insulin analog are insulin lispro, insulin glulisine and insulin aspart. Insulin analogs are commercially available and there are three FDA-approved fast acting insulin analogs on the market: insulin aspart (e.g., commercially available under the tradename NovoLog®/NovoRapid®), insulin glulisine (e.g., commercially available under the tradename Apidra®), and insulin lispro (e.g., commercially available under the tradename Humalog®). Insulin aspart is a single amino acid change from proline to aspartic acid in residue B28 of insulin. Insulin glulisine has a lysine in place of the asparagine in position B3 of insulin and a glutamate in place of lysine in position B29 of insulin. Insulin lispro reverses the order of the penultimate lysine and proline on the C-terminal of the B chain of insulin.

Insulin and its analogs are stored either as lyophilized powder for reconstitution with an aqueous diluent on use or as ready to use aqueous solutions. Both require refrigeration during storage, due to thermal instability.

In an embodiment of the present invention, any pharmaceutically acceptable vasodilatory agent can be used. It is believed, without being bound by theory, that the vasodilatory agent increases blood flow at the site of injection, thereby, carrying the drug away from the site of injection more effectively. In doing so, the increased blood flow creates better sink conditions that facilitate diffusion of the drug into circulation more rapidly than in the absence of increased blood flow.

Non-limiting examples of suitable vasodilatory agent include a vasodilatory agent that can act by mediating hyperpolarization by blocking calcium ion channels, a cAMP-mediated vasodilatory agent, a cGMP-mediated vasodilatory agent or any combination thereof. In an embodiment of the present invention, the vasodilatory agent that can act by mediating hyperpolarization by blocking calcium ion channels is preferably adenosine, endothelium-derived hyperpolarizing factor, a phosphodiesterase type 5 (PDES) inhibitor, a potassium channel opener or any combination thereof. More preferably, the vasodilatory agent that can act by mediating hyperpolarization by blocking calcium ion channels is adenosine. In an embodiment of the present invention, the cAMP-mediated vasodilatory agent is preferably prostacyclin, forskolin or any combination thereof. More preferably, the cAMP-mediated vasodilatory agent is prostacyclin. In an embodiment of the present invention, the cGMP-mediated vasodilatory agent is preferably nitroglycerin, a nitric oxide forming agent, amyl nitrite, nitroprusside or any combination thereof. More preferably, the cGMP-mediated vasodilatory agent is nitroglycerin.

The vasodilatory agent is present in an amount effective to increase the absorption of the therapeutic peptide. In an embodiment of the present invention, the vasodilatory agent is present in an amount of about 0.1 to about 50, more preferably, about 0.5 to about 25, even more preferably, about 1 to about 10 mg/mL.

In a preferred embodiment, the vasodilatory agent is nitroglycerin. In an embodiment of the present invention, nitroglycerin is present in the formulation at a concentration of about 0.1 to about 10, more preferably, about 0.5 to about 7.5, even more preferably, about 1 to about 5 mg/mL.

It is believed that nitroglycerin has a water solubility <1 mg/mL. The inventors of the present invention found that it is possible to increase the water solubility of nitroglycerin by the addition of a suitable surfactant, a co-solvent or a combination thereof. In an embodiment of the present invention, the vasodilatory agent is combined with a surfactant, a co-solvent or a combination thereof. Non-limiting examples of suitable surfactant include polyethylene glycol, a pluronic, a polysorbate or a polaxamer. Non-limiting examples of suitable co-solvent include glycerin, propylene glycol, ethylene glycol, and polyethylene glycol. In an embodiment of the present invention, the surfactant, the co-solvent or a combination thereof is present at a concentration of about 1 to about 25, more preferably, about 1 to about 10, even more preferably, about 1 to about 5 mg/mL.

In a preferred embodiment, the surfactant is PEG3350 (polyethylene glycol with an average molecular weight of 3,350 Da). The inventors of the present invention found that the presence of 20% aqueous solution of PEG3350 increased the soluble concentration of nitroglycerin in an insulin analog injectable formulation to 2 mg/mL. Additionally, the inventors also found that nitroglycerin went into solution more quickly in the presence of PEG3350 than in its absence.

The inventors also found that the presence of a 5% aqueous solution of Tween 80 increased the soluble concentration of nitroglycerin to about 4.5 mg/mL.

The inventors also found that the presence of a 5% aqueous solution of Pluronic F127 increased the soluble concentration of nitroglycerin to about 3.8 mg/mL.

In a preferred embodiment, the co-solvent is propylene glycol, low molecular weight polyethylene glycol less than about 600 Daltons in average molecular weight, glycerin, or any combination thereof.

In an embodiment of the present invention, the rapid acting injectable formulation may optionally contain an antimicrobial agent, a peptide stabilizing excipient, at least one injectable pH altering agent, at least one osmolarity altering agent, a charge masking agent, or a combination thereof.

In an embodiment of the present invention, the rapid acting injectable formulation may optionally contain at least one charge masking agent. It is believed, without being bound by theory, that the charge masking agent promotes the monomer/dimer state of the therapeutic peptide. Non-limiting examples of the charge masking agent include a citrate, a diketopiperazine, a diketopiperazine derivative, ethylenediaminetetraacetic acid (EDTA), di-arginine piperazine, a di-arginine piperazine salt, a di-arginine piperazine isomer, a di-arginine piperazine ester and any combination thereof. The charge masking agent is present at a concentration of about 1 to about 50, more preferably, about 1 to about 25, even more preferably, about 2 to about 20 mg/mL. In a preferred embodiment, the charge masking agent is a citrate, such as, sodium citrate. In an embodiment of the present invention, sodium citrate is present in an amount of about 1 to about 50, more preferably about 1 to about 25, most preferably about 1 to about 10 mg/mL.

The antimicrobial agent is present at a concentration of about 0.1 to about 20, more preferably, about 1 to about 10, even more preferably, about 1 to about 10 mg/mL. Non-limiting examples of the antimicrobial agent include metacresol and m-cresol. Preferably, the antimicrobial agent is metacresol, which is present at a concentration of about 3.15 mg/mL.

The peptide stabilizing excipients, the injectable pH altering agents, and osmolarity altering agents are disclosed in U.S. Provisional Application No. 61/761,545, which is herein incorporated by reference in its entirety.

Specific embodiments of the invention will now be demonstrated by reference to the following examples. It should be understood that these examples are disclosed by way of illustrating the invention and should not be taken in any way to limit the scope of the present invention.

### EXAMPLES

In an embodiment of the present invention, excipients are dissolved and then the therapeutic peptide is added. In another embodiment, one or a combination of vasodilatory agents along with a surfactant, a co-solvent or a combination thereof is added directly to a formulated therapeutic peptide product.

### Example 1

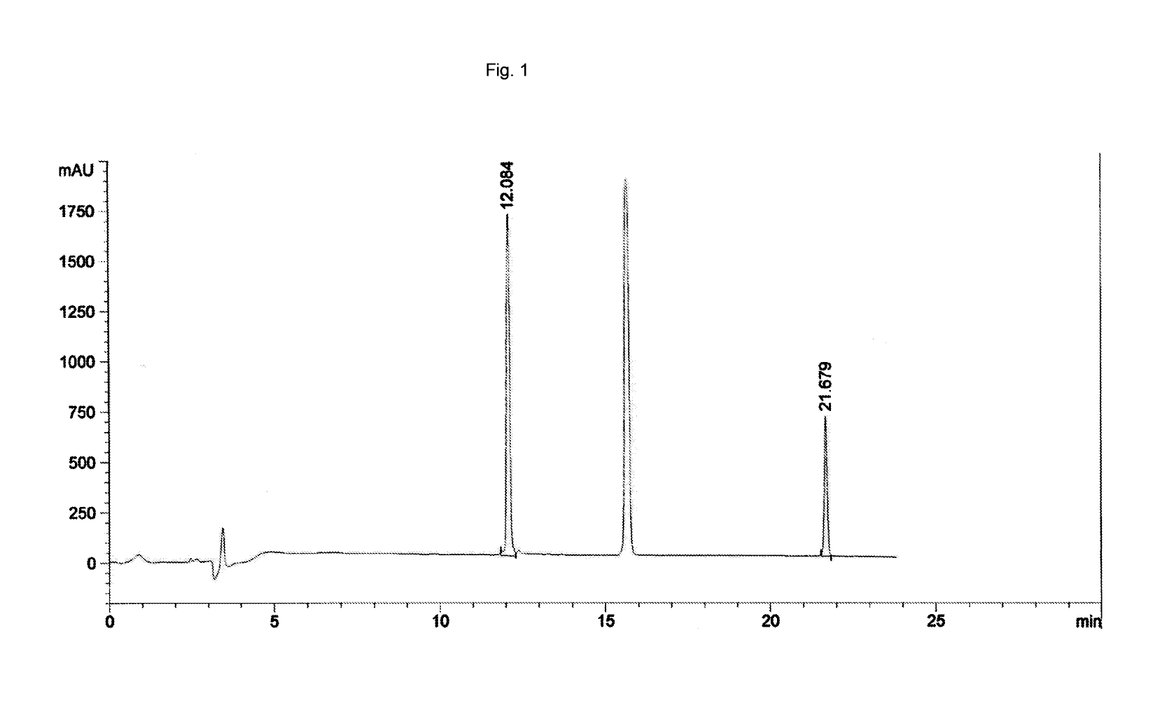

25 nitroglycerin tablets, which are typically administered sub-lingualy, were placed in 5 mL of Humalog® (insulin lispro) aqueous solution overnight on an orbital shaker. The resultant suspension was centrifuged and the supernatant withdrawn to become the test article. The insulin lispro and nitroglycerin concentrations were measured by HPLC. The results are depicted in FIG. 1. The insulin lispro concentration was determined to be 95 IU/mL and the nitroglycerin content was determined to be 1.2 mg/mL. The nitroglycerin peak was found at an elution time of 21.7 minutes in this chromatogram and the insulin lispro peak was found at an elution time of 12.1 minutes in the chromatogram.

### Example 2

50 nitroglycerin tablets were placed in 5 mL of Humalog® aqueous solution overnight on an orbital shaker to see if this will result in an increase in the concentration of nitroglycerin. The final concentration as measured by HPLC was still ˜1 mg/mL. Therefore, the inventors believed that nitroglycerin has reached its solubility limit in the presence of the soluble excipients contained in the nitroglycerin sub-lingual tablets at ˜1 mg/mL. This concentration appears to be sufficient to increase the speed of the absorption of s.c. Humalog®, as demonstrated in diabetic swine experiments detailed below. Nitroglycerin may be sufficiently water soluble to enable enough to go into solution in Humalog® or other peptide hormone drug product formulations of interest as they are currently formulated.

### Example 3

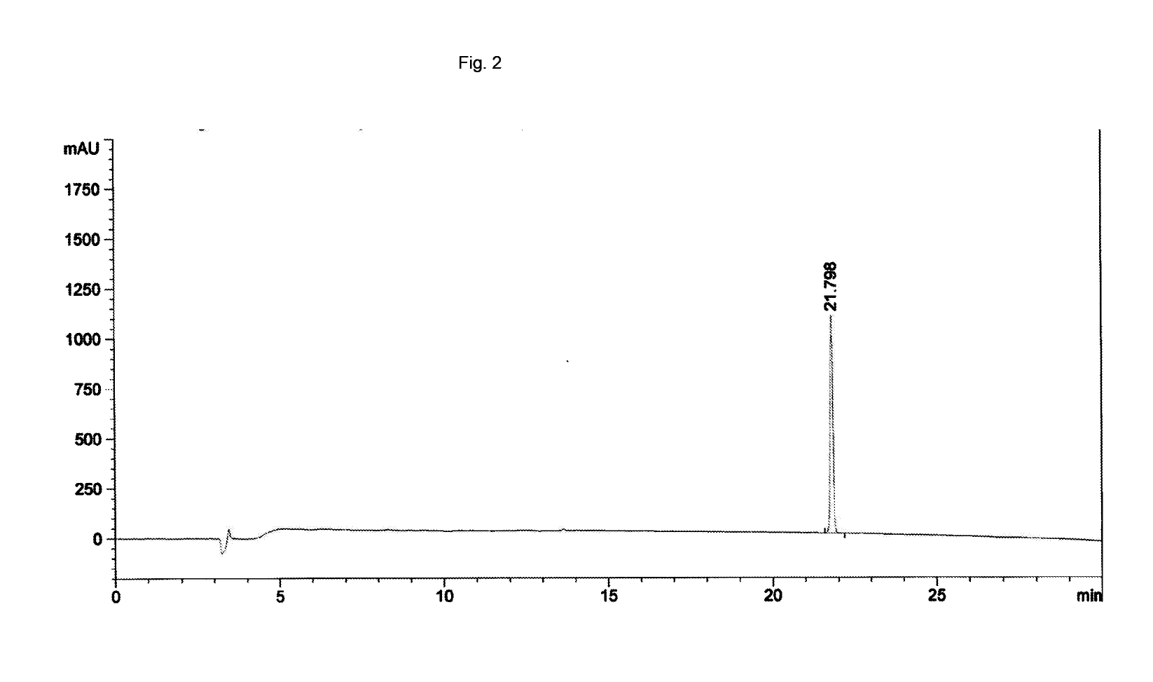

In order to increase the aqueous solubility of nitroglycerin, as noted above, a 20% aqueous solution of PEG3350 was added to the nitroglycerin tablets. The soluble concentration of nitroglycerin in the resultant solution increased to 2 mg/mL. The corresponding HPLC is depicted in FIG. 2. Also as noted above, nitroglycerin went into solution more quickly in the presence of PEG3350 than in its absence.

### COMPARATIVE STUDY

### Diabetic Swine Experiments

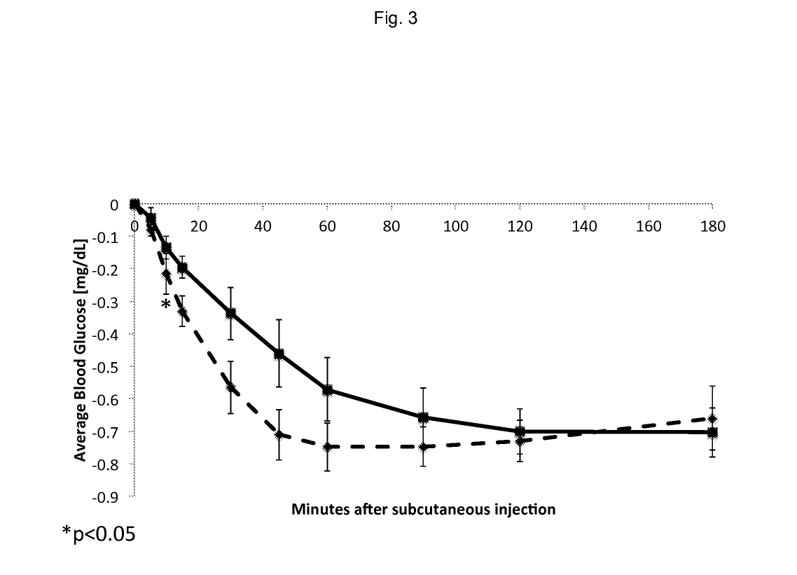

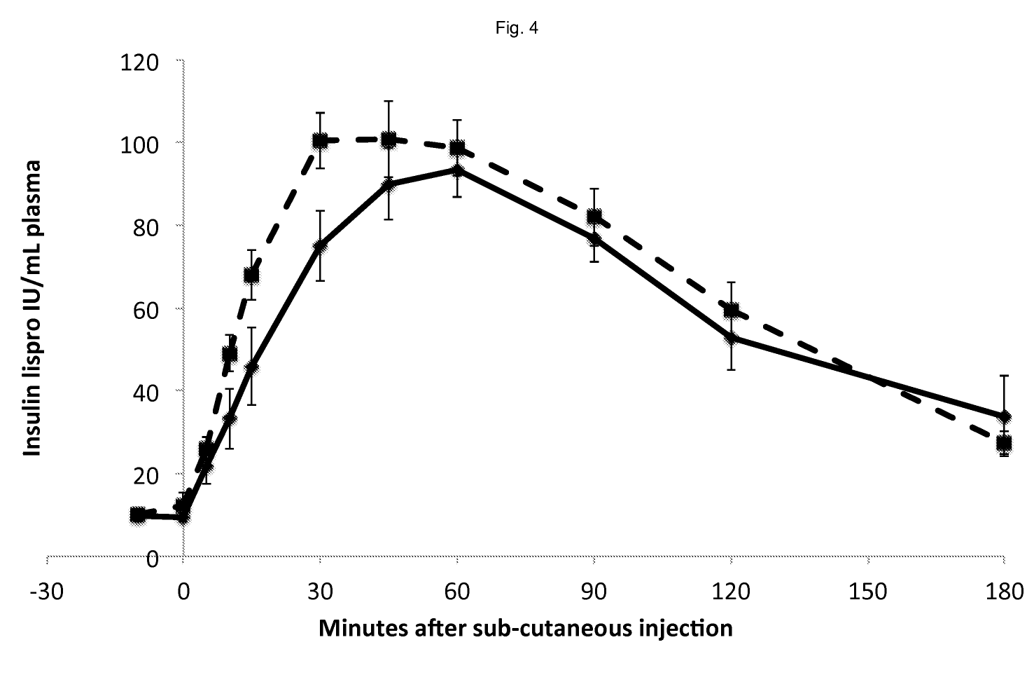

Formulation of insulin lispro with 1.6 mg/mL nitroglycerin in accordance with an embodiment of the present invention and a comparative formulation of insulin lispro without any nitroglycerin (commercially available Humalog® formulation) were administered to each of four diabetic swine twice in a cross-over design with at least 24 hours recovery between doses. Glucose levels were measured as a pharmacodynamic indicator of insulin activity. An increased insulin absorption yields lower blood glucose levels. A plot of the average blood glucose concentration [mg/dL] as function of time for a comparative insulin lispro formulation and for an embodiment of the present invention is shown in FIG. 3. Plasma concentration of insulin lispro was measured by insulin ELISA. A plot of the insulin lispro IU/mL plasma concentration as function of time for a comparative insulin lispro formulation and for an embodiment of the present invention is shown in FIG. 4. In FIG. 3 and FIG. 4, the solid lines with squares depict the results of the comparative insulin lispro formulation without nitroglycerin and the dashed black line with diamonds depict the results of insulin lispro formulation with 1.6 mg/mL nitroglycerin.

As seen in FIG. 3, the Tmax of the pharmacodynamic effect of the insulin lispro formulation of an embodiment of the present invention containing nitroglycerin is 60 minutes as measured in diabetic swine, which is 50% of the about 120 minute Tmax measured for the comparative insulin lispro formulation without nitroglycerin. As seen in FIG. 4, the Tmax of insulin lispro as measured by ELISA for the insulin lispro formulation of an embodiment of the present invention containing nitroglycerin is also 50% of that measured for the comparative insulin lispro formulation without nitroglycerin in the same diabetic swine (30 minutes as compared to 60 minutes). Additionally, the inclusion of nitroglycerin into the insulin lispro formulation increased the area under the insulin lispro concentration versus time pharmacokinetics curve (bioavailability) shown in FIG. 4 by 13% as calculated by the trapezoidal method for area under the curve.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed is:

1. A rapid acting injectable formulation comprising a therapeutic peptide and a vasodilatory agent,
wherein the therapeutic peptide has a molecular weight of greater than about 500 Daltons, and
the vasodilatory agent is present in an amount effective to increase the absorption of the therapeutic peptide.

2. The rapid acting injectable formulation according to claim 1, wherein the therapeutic peptide is selected from a group consisting of insulin, insulin analog, parathyroid hormone (PTH), glucagon, C-peptide, calcitonin, parathyroid hormone, human growth hormone, and incretins.

3. The rapid acting injectable formulation according to claim 1, wherein the therapeutic peptide is insulin or insulin analog.

4. The rapid acting injectable formulation according to claim 3, wherein the insulin analog is selected from the group consisting of insulin lispro, insulin glulisine and insulin aspart.

5. The rapid acting injectable formulation according to claim 1, wherein the vasodilatory agent is selected from the group consisting of a vasodilatory agent that can act by mediating hyperpolarization by blocking calcium ion channels, a cAMP-mediated vasodilatory agent, a cGMP-mediated vasodilatory agent and any combination thereof.

6. The rapid acting injectable formulation according to claim 1, wherein the vasodilatory agent is selected from the group consisting of nitroglycerin, a nitric oxide forming agent, amyl nitrite, nitroprusside, endothelium-derived hyperpolarizing factor, forskolin, a phosphodiesterase type 5 (PDES) inhibitor, a potassium channel opener, adenosine, prostacyclin, and any combination thereof.

7. The rapid acting injectable formulation according to claim 1, wherein the vasodilatory agent is nitroglycerin.

8. The rapid acting injectable formulation according to claim 7, wherein the nitroglycerin is present at a concentration of less than about 10 mg/mL.

9. The rapid acting injectable formulation according to claim 1, wherein the vasodilatory agent is combined with a surfactant, a co-solvent or a combination thereof.

10. The rapid acting injectable formulation according to claim 9, wherein the surfactant is selected from a group consisting of polyethylene glycol, a pluronic, a polysorbate or a polaxamer and the co-solvent is selected from the group consisting of glycerin, propylene glycol, ethylene glycol, and polyethylene glycol.

11. The rapid acting injectable formulation according to claim 10, wherein the polyethylene glycol is PEG3350.

12. The rapid acting injectable formulation according to claim 1, wherein the vasodilatory agent is combined with at least one charge masking agent.

13. The rapid acting injectable formulation according to claim 12, wherein the charge masking agent is selected from the group consisting of a citrate, a diketopiperazine, a diketopiperazine derivative, ethylenediaminetetraacetic acid (EDTA), di-arginine piperazine, a di-arginine piperazine salt, a di-arginine piperazine isomer, a di-arginine piperazine ester and any combination thereof.

14. The rapid acting injectable formulation according to claim 1, wherein the formulation further comprises an antimicrobial agent, a peptide stabilizing excipient, at least one injectable pH altering agent, at least one osmolarity altering agent, a citrate, or any combination thereof.

15. The rapid acting injectable formulation according to claim 3, wherein the vasodilatory agent is nitroglycerin.

16. A method of increasing absorption of a therapeutic peptide by using a rapid acting injectable formulation comprising a therapeutic peptide and a vasodilatory agent,
wherein the therapeutic peptide has a molecular weight of greater than about 500 Daltons, and
the vasodilatory agent is present in an amount effective to increase the absorption of the therapeutic peptide.

17. The method of increasing absorption of a therapeutic peptide according to claim 16, wherein the therapeutic peptide is selected from a group consisting of insulin, insulin analog, parathyroid hormone (PTH), glucagon, C-peptide, calcitonin, parathyroid hormone, human growth hormone, and incretins.

18. The method of increasing absorption of a therapeutic peptide according to claim 16, wherein the therapeutic peptide is insulin or insulin analog.

19. The method of increasing absorption of a therapeutic peptide according to claim 18, wherein the insulin analog is selected from the group consisting of insulin lispro, insulin glulisine and insulin aspart.

20. The method of increasing absorption of a therapeutic peptide according to claim 16, wherein the vasodilatory agent is selected from the group consisting of a vasodilatory agent that can act by mediating hyperpolarization by blocking calcium ion channels, a cAMP-mediated vasodilatory agent, a cGMP-mediated vasodilatory agent and any combination thereof.

21. The method of increasing absorption of a therapeutic peptide according to claim 16, wherein the vasodilatory agent is selected from the group consisting of nitroglycerin, a nitric oxide forming agent, amyl nitrite, nitroprusside, endothelium-derived hyperpolarizing factor, forskolin, a phosphodiesterase type 5 (PDE5) inhibitor, a potassium channel opener, adenosine, prostacyclin, and any combination thereof.

22. The method of increasing absorption of a therapeutic peptide according to claim 16, wherein the vasodilatory agent is nitroglycerin.

23. The method of increasing absorption of a therapeutic peptide according to claim 22, wherein the nitroglycerin is present at a concentration of less than about 10 mg/mL.

24. The method of increasing absorption of a therapeutic peptide according to claim 16, wherein the vasodilatory agent is combined with a surfactant, a co-solvent or a combination thereof.

25. The method of increasing absorption of a therapeutic peptide according to claim 24, wherein the surfactant is selected from a group consisting of polyethylene glycol, a pluronic, a polysorbate or a polaxamer.

26. The method of increasing absorption of a therapeutic peptide according to claim 25, wherein the polyethylene glycol is PEG3350.

27. The method of increasing absorption of a therapeutic peptide according to claim 16, wherein the vasodilatory agent is combined with at least one charge masking agent.

28. The method of increasing absorption of a therapeutic peptide according to claim 27, wherein the charge masking agent is selected from the group consisting of a citrate, a diketopiperazine, a diketopiperazine derivative, ethylenediaminetetraacetic acid (EDTA), di-arginine piperazine, a di-arginine piperazine salt, a di-arginine piperazine isomer, a di-arginine piperazine ester and any combination thereof.

29. The method of increasing absorption of a therapeutic peptide according to claim 16, wherein the formulation further comprises an antimicrobial agent, a peptide stabilizing excipient, at least one injectable pH altering agent, at least one osmolarity altering agent, a citrate, or any combination thereof.

30. The method of increasing absorption of a therapeutic peptide according to claim 18, wherein the vasodilatory agent is nitroglycerin.
